(19) United States
(12) Patent Application Publication
(10) Pub. No.: US 2024/0122488 A1
(43) Pub. Date: Apr. 18, 2024

(54) Wearable Apparatus For Measuring Blood Pressure Signal

(71) Applicant: Hong Kong Centre for Cerebro-Cardiovasculer Health Engineering Limited, Hong Kong (CN)

(72) Inventors: Yuanting ZHANG, Hong Kong (CN); Zijun Liu, Hong Kong (CN); Zhibo Fu, Hong Kong (CN)

(21) Appl. No.: 18/070,493

(22) Filed: Nov. 29, 2022

(30) Foreign Application Priority Data
Oct. 13, 2022 (CN) 202211254761.0

(51) Int. Cl. A61B 5/021; A61B 5/00; A61B 5/024

(52) U.S. Cl. CPC A61B 5/02125; A61B 5/02416; A61B 5/6817; A61B 5/7203

(57) ABSTRACT

The present invention relates to a wearable apparatus for measuring blood pressure signal. The invention relates to the field of blood pressure measuring technology. The wearable apparatus for measuring blood pressure signal comprises an earphone body, an optical sensor and a processing module, wherein the optical sensor is arranged at the earphone body to obtain a multi-wavelength photoplethysmogram (PPG) signal at an ear; the wavelength of the PPG signal is related to the skin attribute of its location; the processing module is used for obtaining a physiological parameter according to the multi-wavelength PPG signal, the physiological parameter comprises a blood pressure signal. Placing the earphone body on the ear is convenient to a user without affecting normal activities of the user so as to perform a long-term monitoring of the user. At the same time, since the wavelength of the PPG signal is related to the skin attribute of its location, by arranging the optical sensor to obtain a plurality of PPG signals can ensure the blood pressure signal can be obtained only by monitoring the ear. Further, the position between the ear and the heart is relatively stable which improves the monitoring accuracy.

### FIELD OF THE INVENTION

The present invention relates to the technical field of blood pressure monitoring, specifically, the invention relates to a wearable apparatus for blood pressure signal monitoring.

### BACKGROUND OF THE INVENTION

As people pay more and more attention to the physiological health condition, the application of wearable apparatus is becoming increasingly popular among users, which means that users can monitor and count various physiological parameters (for example, blood pressure) in real-time through wearable apparatus, so that they can have an intuitive awareness of the body conditions and then take corresponding measures promptly. Considering current techniques, the blood pressure is calculated through pulse transition time (PTT), to acquire this PTT, the existing wearable devices often need simultaneous measurement on different locations on the body (for example, wrist and finger), which makes these devices inconvenient to wearable thus cannot be used for long-term measurement.

### OBJECTS OF THE INVENTION

An object of the present invention is to provide a wearable apparatus for blood pressure signal monitoring.

The above object is met by the combination of features of the main claim; the sub-claims disclose further advantageous embodiments of the invention.

One skilled in the art will derive from the following description other objects of the invention. Therefore, the foregoing statements of object are not exhaustive and serve merely to illustrate some of the many objects of the present invention.

### SUMMARY OF THE INVENTION

Embodiments of the present invention provide a wearable apparatus for monitoring blood pressure, which can solve the problem of prolonged monitoring of blood pressure. The described technical solution is as follows:

One aspect of the present invention provides a wearable apparatus for blood pressure measurement, comprising earphone body, optical sensor, and data processing module, wherein the optical sensor is arranged at the earphone body, the optical sensor is for obtaining multi-wavelength photoplethysmogram (PPG) signal, wherein the wavelengths are selected according to the skin property of the sensor location.

The data processing module is for obtaining physiological parameters based on the multi-wavelength PPG signal, the physiological parameters include blood pressure signal.

As an optional embodiment, the multi-wavelength PPG signal includes:
Signal 1, wherein Signal 1 contains pulsatile information from the capillary layer under the skin in the ear location.
Signal 2, wherein Signal 2 contains pulsatile information from the capillary layer and the arteriolar layer under the skin in the ear location.
Signal 3, wherein Signal 3 contains pulsatile information from the capillary layer, the arteriolar layer and the arterial layer under the skin in the ear location.

As an optional embodiment of the wearable apparatus for blood pressure signal measurement, the data processing module comprising:
pre-processing unit for the pre-process of multi-wavelength PPG signal, wherein pre-processing includes filtering and noise reduction processing;
physiological signal estimation unit for inputting pre-processed multi-wavelength PPG signal to pre-constructed physiological system mathematical model or deep learning algorithm and calculating physiological signals, wherein the deep learning algorithms were trained using multi-wavelength PPG signal as input sample and blood pressure signal as label.

As an optional embodiment of the wearable apparatus for blood pressure signal measurement, sensors also include accelerometer, wherein the accelerometer is arranged at the earphone body; the pre-processing unit is used for performing the noise reduction processing according to an accelerometer signal collected by the accelerometer.

As an optional embodiment of the wearable apparatus for blood pressure signal measurement, sensors also include pressure sensor, wherein the pressure sensor is optionally arranged at the earphone body; the physiological parameter estimation unit is used for calibrating the physiological system mathematical model according to the pressure signal collected at different body positions of the user by the pressure sensor.

As an optional embodiment of the wearable apparatus for blood pressure signal measurement, sensors also include physiological sensor, wherein the physiological signal estimation unit uses electric signal acquired from the ear location by the physiological sensor to calibrate the mathematical model.

As an optional embodiment of the wearable apparatus for blood pressure signal measurement, the system includes a communication module, wherein the communication module is arranged at the earphone body.

When the data processing module is arranged at the earphone body, the communication module is for sending multi-wavelength PPG signal to data processing module, and the communication module is for transmission of physiological signal calculated by the data processing module.

When the data processing module is arranged outside of the earphone body, the communication module is for transmission of multi-wavelength PPG signal to the data processing module.

As an optional embodiment of the wearable apparatus for blood pressure signal measurement, the physiological signal includes heart rate and blood oxygen level; the blood pressure signal includes mean blood pressure, diastolic pressure, systolic pressure, beat-to-beat blood pressure and tonoarteriogram (TAG) information.

As an optional embodiment of the wearable apparatus for blood pressure signal measurement, the earphone body at least one of an in-ear earphone body and an ear-hook earphone body.

The technical embodiment of the present invention provides a wearable apparatus for detecting physiological parameters using the earphone body placed on the ear location, which is convenient for the user to wear without affecting the user's everyday activities, thus enabling the user to be monitored for a long time. In addition, because the light wavelength of the PPG sensor is arranged according to the skin property of the sensing site, the sensor is able to sense PPG signal that covers pulsatile information of different blood vessel layers under the skin, enabling acquisition of blood pressure signal from only ear location, which makes the device easy to wear. Moreover, compared with other locations on human body, the ear is rich in blood vessels, and the position between the ear and the heart is relatively fixed with less movement, which can improve monitoring accuracy.

### BRIEF DESCRIPTION OF THE DRAWINGS

The foregoing and further features of the present invention will be apparent from the following description of preferred embodiments which are provided by way of example only in connection with the accompanying figures, of which:

FIG. 1 shows a schematic diagram of the installation location of the earphone body;

FIG. 2 shows a schematic diagram of the wearable apparatus for monitoring blood pressure;

FIG. 3 shows a schematic diagram of the structure of the optical sensor;

FIG. 4 shows a schematic diagram of the structure of the connected optical sensor and the earphone body;

FIG. 5 shows a schematic diagram of the structure of the connected physiological sensor and the earphone body;

FIG. 6 shows a schematic diagram of the structure of the earphone body;

FIG. 7 shows a schematic diagram of an example of the PPG signal; and

FIG. 8 shows a schematic diagram of the structure of using blood pressure monitor to calibrate the wearable apparatus for monitoring blood pressure.

### DESCRIPTION OF PREFERRED EMBODIMENTS

Embodiments of the present invention are described in connection with the following figures in the present invention. It should be understood that the embodiments set forth below in conjunction with the accompanying figures are exemplary descriptions to explain the technical solutions of the embodiments of the present invention and do not constitute a limitation of the technical solutions of the embodiments of the present invention.

It will be understood by technicians in the art unless expressly stated, the singular forms “one,” “a”, “said,” and “the” as used herein “may also include the plural form. It should be understood that when we refer to a component being “connected” or “coupled” to another element, the one component may be directly connected or coupled to the other component, or it may refer to the one component and the other component being connected through an intermediate component. The term “and/or” as used herein refers to at least one of the items defined by the term; for example, “A and/or B” may be implemented as “A” or as “B” or “A and B”.

In order to make the purpose, technical solutions and advantages of this invention clearer, the following will be described in further detail in conjunction with the accompanying figures for the implementation of this invention.

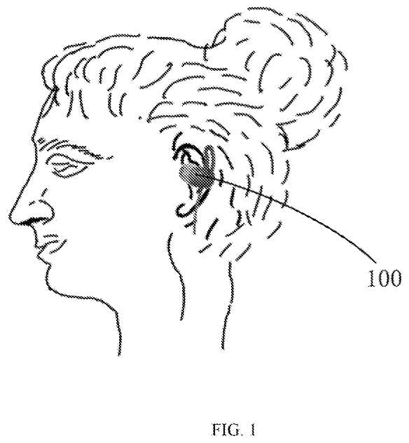

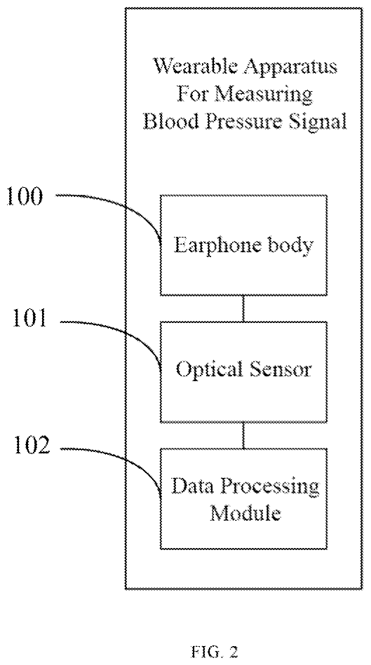

Embodiments of the present invention provide a wearable apparatus for monitoring blood pressure, as shown in FIGS. 1 and 2. FIG. 1 exemplarily illustrates a schematic structure of installation location of the earphone body 100, FIG. 2 exemplarily illustrates a schematic structure of wearable apparatus for monitoring blood pressure, the wearable apparatus for monitoring blood pressure comprising earphone body 100, optical sensor 101 and data processing module 102, optical sensor 101 is arranged at earphone body 100, optical sensor 101 is for acquiring multi-wavelength PPG signal on the ear location; wherein the wavelength of the PPG sensor is related to the skin property of the sensing location; data processing module 102 is for acquiring physiological signal based on multi-wavelength PPG signal, wherein the physiological signal includes blood pressure signal.

It can be understood that the earphone body 100 is put on the ear, which is convenient for the user to wear without affecting the everyday activities of the user, so that the user can be monitored for a long time by optical sensor 101 and data processing module 102. In addition, compared with other parts of the human body, the ear is rich in vascular distribution and less mobile and relatively fixed position between the heart, which can improve monitoring accuracy.

Optionally, when there are two earphone bodies 100, the two earphone bodies 100 can communicate wirelessly with each other, which is convenient for the user to wear without affecting the everyday activities of the user. In other embodiments, the two earphone bodies 100 can be connected through chain or ear-hook, which is convenient for the user to store the two earphone bodies 100.

Wherein, as illustrated by FIG. 3, FIG. 3 shows a schematic diagram of the structure of the optical sensor, the optical sensor 101 includes photodetector 1011 and multiple light emitter 1012, light emitter 1012 is for emitting monochromatic light onto the ear, photodetector 1011 is for collecting the reflected or transmitted light from the ear to get PPG signal, multiple light emitter 1012 emit light with different wavelength, enabling the optical sensor 101 to generate multi-wavelength light.

It should be explained that the PPG signal, also known as plethysmography signal, is detected by the PPG sensor to obtain the change curve of blood volume over time. Multi-wavelength composite light is light composed of a variety of different wavelengths of monochromatic light, because there are differences in the absorption, attenuation and other effects of blood on each wavelength, so when the optical sensor 101 generates multi-wavelength composite light (i.e., light composed of a variety of different wavelengths of monochromatic light) it can obtain a multi-wavelength PPG (MWPPG) signal. Compared to the prior art by placing additional devices on other body parts of the user to obtain blood pressure signal, using the optical sensor 101 to generate multi-wavelength light for acquiring blood pressure signal can greatly reduce the number of parts, making the miniaturization of optical sensors conducive to integration and improving user experience.

Preferably, multiple light emitter 1012 can be arranged at one side of the photodetector 1011, or can be arranged at both sides of the photodetector 1011, or can be arranged around the photodetector 1011, this embodiment have no limit on the arrangement of the light emitter 1012.

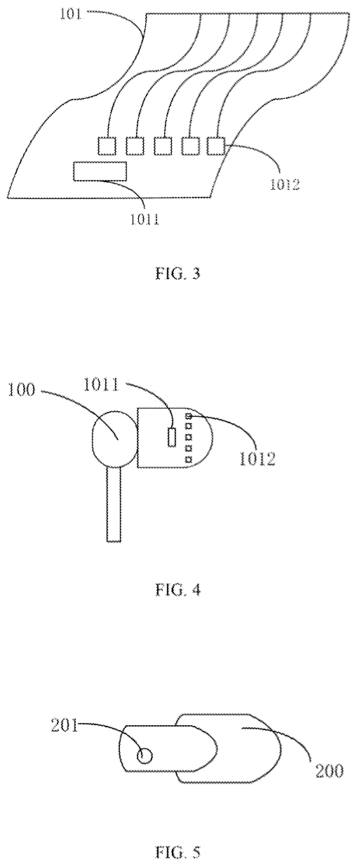

Preferably, as illustrated by FIG. 4, FIG. 4 shows a schematic diagram of the structure of the connected optical sensor and the earphone body, the optical sensor 101 can be arranged at the sound tube of the earphone body 100, which means when earphone body 100 is in the form of in-ear earphone, the optical sensor 101 is located in the ear canal when the user wears the earphone.

For one aspect, because the skin properties (skin thickness and blood vessel layer under the skin) on different ear location (ear canal, antihelix, auricle and posterior auricular) are different, thinner skin have less blood vessel layers, some skin only have capillary layer, some skin have capillary layer and arteriolar layer, some skin have capillary layer, arteriolar layer and arterial layer, so the acquired PPG signal will be different if the optical sensor 101 is put on different locations on the ear.

For another aspect, because monochromatic light with different wavelength can penetrate to different depth under the skin, the acquired PPG signal is different, for example, blue light with 460 nm wavelength mainly penetrates to capillary layer, yellow light with 593 nm wavelength mainly penetrates to arteriolar layer, near infrared light with 940 nm wave length can penetrate to arterial layer, so if the light emitter 1012 in the optical sensor 101 emit light with different wavelengths, the acquired PPG signal is different.

Exemplarily, the relationship between the PPG signal and the pulsatile information of different blood vessel layers is as follows:

B_PPG≈f(V_{c})+f(Vₘ)

Wherein, B_PPG represents the PPG signal of blue light with 460 nm wavelength, f(V_{c}) represents pulsatile information of capillary layer, f(Vₘ) represents motion artifact (noise in PPG signal).

Y_PPG≈f(V_{c})+f(V₀)+f(Vₘ)

Wherein, Y_PPG represents the PPG signal of yellow light with 593 nm wavelength, f(V_{c}) represents pulsatile information of capillary layer, f(V₀) represents pulsatile information of arteriolar layer, f(Vₘ) represents motion artifact (noise in PPG signal).

IR_PPG≈f(V_{c})+f(V₀)+f(Vₐ)+f(Vₘ)

Wherein, IR_PPG represents the PPG signal of near infrared light with 940 nm wavelength, f(V_{c}) represents pulsatile information of capillary layer, f(V₀) represents pulsatile information of arteriolar layer, f(Vₐ) represents pulsatile information of arterial layer, f(Vₘ) represents motion artifact (noise in PPG signal).

It can be understood that the earphone body 100 is put on the ear, which is convenient for the user to wear without affecting the everyday activities of the user, so that the user can be monitored for a long time. In addition, because the light wavelength of the PPG sensor is arranged according to the skin property of the sensing site, the sensor is able to sense PPG signal that covers pulsatile information of different blood vessel layers under the skin, enabling acquisition of blood pressure signal from only ear location, which makes the device easy to wear. Moreover, compared with other locations on human body, the ear is rich in blood vessels, and the position between the ear and the heart is relatively fixed with less movement, which can improve monitoring accuracy.

Based on each of the above embodiments, as an optional embodiment, the multi-wavelength PPG signal includes:
Signal 1, wherein Signal 1 contains pulsatile information from the capillary layer under the skin in the ear location.
Signal 2, wherein Signal 2 contains pulsatile information from the capillary layer and the arteriolar layer under the skin in the ear location.
Signal 3, wherein Signal 3 contains pulsatile information from the capillary layer, the arteriolar layer and the arterial layer under the skin in the ear location.
   Preferably, blue light with 460 nm wavelength mainly penetrates to capillary layer, so the optical sensor can acquire Signal 1 that contains pulsatile information from the capillary layer through blue light; yellow light with 593 nm wavelength mainly penetrates to arteriolar layer, so the optical sensor can acquire Signal 2 that contains pulsatile information from the capillary layer and the arteriolar layer through yellow light; near infrared light with 940 nm wave length can penetrate to arterial layer, so the optical sensor can acquire Signal 3 that contains pulsatile information from the capillary layer, the arteriolar layer and the arterial layer through near infrared light. When the optical sensor emits mixed light that contains blue light, yellow light and near infrared light, the acquired multi-wavelength PPG signal contains Signal 1, Signal 2 and Signal 3.

Based on each of the above embodiments, as an optional embodiment, the data processing module comprising:
pre-processing unit for the pre-process of multi-wavelength PPG signal, wherein pre-processing includes filtering and noise reduction;
physiological signal estimation unit for inputting pre-processed multi-wavelength PPG signal to pre-constructed physiological system mathematical model or deep learning algorithms and calculating physiological signals, wherein the deep learning algorithms were trained using multi-wavelength PPG signal as input sample and blood pressure signal as label.

By implementing a pre-constructed physiological system mathematical model or deep learning algorithm to the physiological signal estimation unit, the system can have a lower response time and the estimation unit can have a higher calculation efficiency.

Preferably, pre-processing methods includes but not limited to re-sampling, smoothing, high-pass filter, low-pass filter, notch filter, wavelet filter and machine learning algorithms, wherein, the machine learning algorithms includes but not limited to principal component analysis, individual component analysis and convolutional neural network.

Optionally, pre-processing unit comprising controller and signal processor, wherein the controller is for controlling the working state of the optical sensor and the signal processor is for pre-processing multi-wavelength PPG signal.

Preferably, signal processor comprising filter, amplifier and analog-to-digital converter, wherein the filter is for filtering noise inside biological signal, the amplifier is for amplifying the biological signal for the analog-to-digital converter to acquire the signal, the analog-to-digital converter is for converting the biological signal to digital signal for the physiological system mathematical model or deep learning algorithms to conduct calculation.

In one embodiment, the physiological signal estimation unit is arranged at the earphone body, to increase the integration level of the wearable apparatus for measuring blood pressure signal. In other embodiments, the physiological signal estimation unit can be arranged outside, meaning that it can be arranged on other wearable apparatus, the measurement accuracy of the blood pressure signal can be improved through interaction with other wearable apparatus.

Based on each of the above embodiments, as an optional embodiment, the wearable apparatus for measuring blood pressure signal also include accelerometer, wherein the accelerometer is arranged at the earphone body; the pre-processing unit is used for performing the noise reduction processing according to an accelerometer signal collected by the accelerometer.

It should be explained that considering that the physiological signal is more accurate when measured while the body is at rest, the pre-processing unit can use the acceleration signal to reduce the noise in the acquired PPG signal, enabling a more accurate measurement of blood pressure signal.

Based on each of the above embodiments, as an optional embodiment, the wearable apparatus for measuring blood pressure signal also includes pressure sensor, wherein the pressure sensor is arranged at the earphone body, the physiological signal estimation unit uses pressure signals acquired while the user is in different body positions to calibrate the mathematical model.

Wherein, the different body position includes the change between standing, crouching, and sitting. The change of body position will affect the blood pressure signal, so the collected pressure signal when the user is indifferent body position can be used to calibrate the physiological system mathematical model, to ensure the accuracy of the blood pressure signal measurement.

Based on each of the above embodiments, as an optional embodiment, the wearable apparatus for measuring blood pressure signal also includes biological sensor, wherein the biological sensor is arranged at the earphone body; the physiological parameter estimation unit is used for calibrating the physiological system mathematical model according to a bio-electrical signal of the ear collected by the biological sensor.

Preferably, the biological sensor is in the form of at least one of the followings: patch type sensor, conductive silicone type sensor.

In addition, the pre-processing unit in the data processing module process the electrophysiological signals to get electrocardiogram (ECG) signal. The physiological signal estimation unit use the combination of ECG and PPG signal to acquire pulse transition time, to further calculate the blood pressure signal, where in this blood pressure signal can be used to calibrate the blood pressure signal derived from the multi-wavelength PPG signal, to ensure the physiological system mathematical model can better adapt to the user.

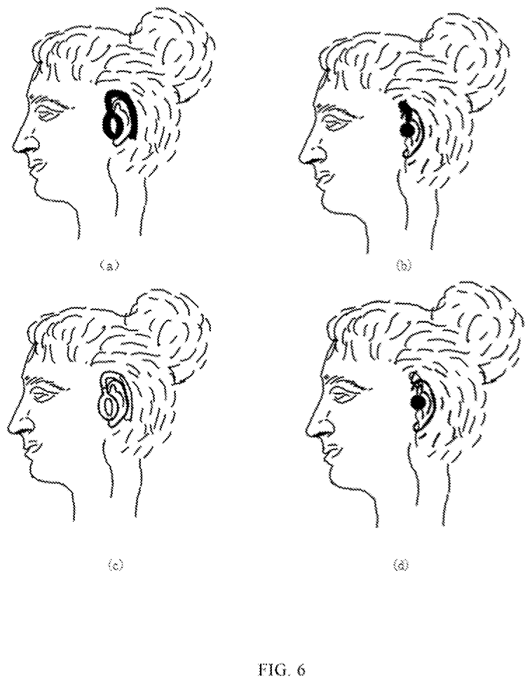

Preferably, as illustrated by FIG. 5, FIG. 5 shows a schematic diagram of the structure of the connected physiological sensor and the earphone body, the earphone body is in the form of in-ear earphone, the biological sensor electrode 201 is arranged at the silicone tip of the earphone 200. In other embodiments, where the earphone body is in the form of ear-hook earphone, the biological sensor electrode can be arranged at the shark-fin shaped part or ear-hook on the earphone body.

Optionally, when there are two earphone bodies, the biological sensor electrode 201 can be arranged at one of the earphone bodies or on both earphone bodies, this embodiment does not limit on the above arrangement.

Based on each of the above embodiments, as an optional embodiment, the wearable apparatus for measuring blood pressure signal also includes communication module, wherein the communication module is arranged at the earphone body.

When the data processing module is arranged at the earphone body, the communication module is for sending the multi-wavelength PPG signal to data processing module, and the communication module is for transmission of the physiological signals processed by data processing module.

When the data processing module is arranged outside of the earphone body, the communication module is for transmitting multi-wavelength PPG signal to data processing module.

Preferably, the communication module includes Bluetooth master control unit, which is for connecting wirelessly to display apparatus to export physiological signals, wherein the display apparatus can be mobile phone, tablet, computer, smart watch, etc.

Based on each of the above embodiments, as an optional embodiment, the wearable apparatus for measuring blood pressure signal also includes power supply module, the power supply module is for supplying power to the system for monitoring physiological signals, to ensure the wearable apparatus for measuring blood pressure signal can operate normally and for a long time.

In one embodiment, when there are two earphone bodies, two power supply units can be arranged at each one of the earphone bodies, or the power supply unit can be arranged at one of the earphone bodies and the two earphone bodies are connected through wire.

It can be understood that, if the earphone body communicate to each other wirelessly, there are two power supply units in each earphone bodies; if the two earphone bodies are connected through chain or ear hook, the power supply unit can be arranged at one of the earphone bodies, and connected to another earphone body through chain or ear-hook that are made with conductive materials (equivalently, conductive wire), to supply power to the other earphone body, the power supply unit can also be arranged at both earphone bodies, this embodiment does not limit such arrangement.

Based on each of the above embodiments, as an optional embodiment, the physiological signal also includes heart rate and blood oxygen level; the blood pressure signal includes mean blood pressure (MBP), systolic blood pressure (SBP), beat-to-beat blood pressure and Tonoarteriogram (TAG).

Wherein, Tonoarteriogram can have a more accurate assessment of cardiovascular system of the user, which ensures the accuracy of assessment of the user health condition.

Based on each of the above embodiments, as an optional embodiment, the earphone body is at least one of an in-ear earphone body and an ear-hook earphone body.

The earphone body is arranged as an in-ear earphone body or an ear-hook earphone body depending on the need of different user. It should be explained that this embodiment does not limit the structure or shape of the earphone body if the user can easily wear them.

Specifically, as illustrated by FIG. 6, FIG. 6 shows a schematic diagram of the structure of the earphone body, in (a)˜(d), the earphone body is ear-hook earphone body, but in (a) and (c) the earphone body is not in-ear earphone, in (b) and (d), the earphone body is in-ear earphone, which means that in (a) and (c) the earphone body is ear-hook earphone body, in (b) and (d), the earphone body is both in-ear earphone body and ear-hook earphone body.

Based on each of the above embodiments, as an optional embodiment, the wearable apparatus for measuring blood pressure signal include earphone body, optical sensor and data processing unit, the optical sensor is for obtaining multi-wavelength photoplethysmogram (PPG) signal, wherein the wavelengths are selected according to the skin property of the sensor location; the pre-processing unit is for performing pre-processing of the multi-wavelength PPG signal, the pre-processing comprising filtering and noise reduction processing; the physiological signal estimation unit is for inputting pre-processed multi-wavelength PPG signal to pre-constructed physiological system mathematical model or deep learning algorithm and calculating physiological signals, where in the physiological signals include blood pressure signal.

In addition, the wearable apparatus for measuring blood pressure signal include accelerometer, pressure sensor and biological sensor, wherein the accelerometer is arranged at the earphone body; the pre-processing unit is used for performing the noise reduction processing according to an accelerometer signal collected by the accelerometer; wherein the biological sensor is arranged at the earphone body; the physiological parameter estimation unit is used for calibrating the physiological system mathematical model according to a bio-electrical signal of the ear collected by the biological sensor; wherein the pressure sensor is optionally arranged at the earphone body; the physiological parameter estimation unit is used for calibrating the physiological system mathematical model according to the pressure signal collected at different body positions of the user by the pressure sensor.

The method of this embodiment comprising:
acquiring multi-wavelength PPG signal from the ear;
sending the multi-wavelength PPG signal to pre-processing unit for pre-processing;
sending the pre-processed multi-wavelength PPG to pre-constructed physiological system mathematical model or deep learning algorithm and calculating physiological signals, wherein the physiological signals include blood pressure signal.

In one embodiment, considering Tonoarteriogram is composed of stable component, which is mean blood pressure and pulsatile component, which is pulse pressure, mean blood pressure and pulse pressure can be derived from PPG signal, meaning that blood pressure signal includes mean blood pressure and pulse pressure, the physiological system mathematical model includes following formulas:

$$MBP = HR * ( {k}_{1} * PTT + {b}_{1} )$$

$$PP = MBP * ( {k}_{2} * \frac{{t}_{τ}}{HP} ) + {b}_{2} )$$

Wherein:
k₁, k₂, b₁ and b₂ are dependent variables, which can be derived by inputting PPG signal in above formulas. Considering CO=SV*HR, cardiac output (CO), Stroke Volume (SV), Pulse wave transit time (PTT) can be derived from PTT, heart rate (HR) can also be derived.

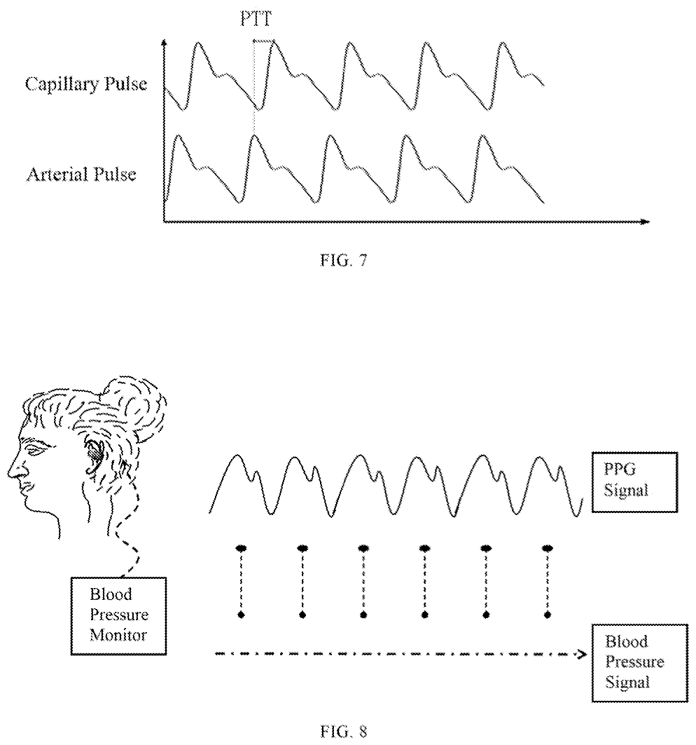

Preferably, heart rate can be calculated from the time difference between two consecutive peaks in blue PPG signal, PTT can be derived from the time difference between peak in different pulsatile signal within the same heart period. FIG. 7 shows a schematic diagram of an example of the PPG signal, wherein PTT is the time difference between peak in capillary pulsatile signal and arterial pulsatile signal.

Considering t_{τ}≈SVR*AC, peripheral vascular resistance (SVR), arterial compliance (AC) can be derived from PPG signal, time constant t_{τ} and DPB can also be derived. In other embodiments, t_{τ} can be derived from the near infrared PPG signal.

In one embodiment, to ensure the wearable apparatus for measuring blood pressure signal can measure blood pressure accurately after a long time, regular calibration to physiological system mathematical model is needed. As illustrated in FIG. 8, FIG. 8 shows a schematic diagram of the structure of using blood pressure monitor to calibrate the wearable apparatus for monitoring blood pressure, the system connect with existing blood pressure monitor through the communication module, the existing blood pressure monitor will take random measurements within a time period, the blood pressure signal is time synchronized with the PPG signal, the data processing unit will use the blood pressure signal acquired from the existing blood pressure monitor to calibrate the parameters, k₁, k₂, b₁ and b₂ in physiological system mathematical model.

Based on each of the above embodiments, as an optional embodiment, the physiological system mathematical model also include following formula:

$$MBP = \frac{k}{PTT} + b$$

Wherein, k and b are dependent variables, PTT can be derived from the combination of ECG and PPG signal.

Thus, when using ECG for calibration, first MBP can be derived from the multi-wavelength PPG signal, second MBP can be derived from the combination of ECG and PPG signal, by comparing the first and second MBP signal, the system determines whether the physiological system mathematical model need calibration. When calibration is needed, the first MBP can be used to calibrate k and b, the second MBP can be used to calibrate k₁, k₂, b₁ and b₂.

Based on each of the above embodiments, as an optional embodiment, the physiological system mathematical model also include following formula:

MBP=f(ΔPTT)

Wherein, ΔPTT is the variation in PTT when the user is in different body position. When the body position is changed, the height difference will cause change in transient pressure, which will also result in change in PTT, meaning ΔPTT=f(h), the height difference can be acquired from accelerometer.

Thus, when using pressure signal for calibration, first MBP can be derived from the multi-wavelength PPG signal, third MBP can be derived from the pressure signal, by comparing the first and third MBP signal, the system determines whether the physiological system mathematical model need calibration. When calibration is needed, the third MBP can be used to calibrate k₁, k₂, b₁ and b₂.

The terms “first”, “second”, “third”, “fourth”, “1”, “2”, etc. (if present) in the specification, claims and the accompanying drawings are used to distinguish similar objects and need not be used to describe a particular order or sequence. It should be understood that the data so used is interchangeable where appropriate so that the embodiments of the present invention described herein can be implemented in an order other than that illustrated or described in the text.

It should be understood that although arrows indicate the individual operational steps in the flowcharts of embodiments of the present invention, the order in which these steps are performed is not limited to the order indicated by the arrows. Unless explicitly stated herein, in some implementation scenarios of embodiments of the present invention, the implementation steps in the respective flowcharts may be performed in other orders as desired. In addition, some or all of the steps in each flowchart may include multiple sub-steps or stages based on actual implementation scenarios. Some or all of these sub-steps or phases may be executed at the exact moment, and each of these sub-steps or phases may also be executed separately at different moments. In the scenario where the execution time is different, the order of execution of these sub-steps or stages can be flexibly configured according to the needs, and this embodiment is not limited in this regard.

It should be noted that for a person of ordinary skill in the art, other similar means of implementation based on the technical ideas of the present invention, without departing from the technical conception of the scheme of the present invention, also fall within the scope of protection of the embodiments of the present invention.

We claim:

1. A wearable apparatus for measuring blood pressure signal, comprising an earphone body, an optical sensor and a processing module; the optical sensor being arranged at the earphone body for obtaining a multi-wavelength photoplethysmogram (PPG) signal at an ear; the wavelength of each PPG signal is related to skin attribute of its location; the processing module is used for obtaining a physiological parameter according to the multi-wavelength PPG signal, wherein the physiological parameter comprises a blood pressure signal.

2. The wearable apparatus for measuring blood pressure signal according to claim 1, wherein the multi-wavelength PPG signal comprises:
a first PPG signal comprising a pulsatile information of a capillary layer of the ear;
a second PPG signal comprising a pulsatile information of the capillary layer and an arteriole layer of the ear;
a third PPG signal comprising a pulsatile information of the capillary layer, the arteriole layer and an arterial layer of the ear.

3. The wearable apparatus for measuring blood pressure signal according to claim 1, wherein the processing module comprises:
a pre-processing unit for performing pre-processing of the multi-wavelength PPG signal, the pre-processing comprising filtering and noise reduction processing;
a physiological parameter estimation unit for inputting the pre-processed multi-wavelength PPG signal into a pre-built physiological system mathematical model or a deep learning model to obtain the physiological parameter; the deep learning model is trained by taking the multi-wavelength PPG signal as an sample and the blood pressure signal as a label.

4. The wearable apparatus for measuring blood pressure signal according to claim 3, further comprising an accelerometer, wherein the accelerometer is arranged at the earphone body; the pre-processing unit is used for performing the noise reduction processing according to an accelerometer signal collected by the accelerometer.

5. The wearable apparatus for measuring blood pressure signal according to claim 3, further comprising a pressure sensor, wherein the pressure sensor is optionally arranged at the earphone body; the physiological parameter estimation unit is used for calibrating the physiological system mathematical model according to the pressure signal collected at different body positions of the user by the pressure sensor.

6. The wearable apparatus for measuring blood pressure signal according to claim 3, further comprising a biological sensor, wherein the biological sensor is arranged at the earphone body; the physiological parameter estimation unit is used for calibrating the physiological system mathematical model according to a bio-electrical signal of the ear collected by the biological sensor.

7. The wearable apparatus for measuring blood pressure signal according to claim 1, further comprising a communication module, wherein the communication module is arranged at the earphone body,
wherein when the processing module is arranged at the earphone body, the communication module is used for transmitting the multi-wavelength PPG signal to the processing module, and the communication module is used for outputting the physiological parameter obtained by the processing module;
wherein when the processing module is arranged to separate from the earphone body, the communication module is used for transmitting the multi-wavelength PPG signal to the processing module.

8. The wearable apparatus for measuring blood pressure signal according to claim 1, wherein the physiological parameter further comprises a heart rate and a blood oxygen saturation level;
wherein the blood pressure signal comprises a mean blood pressure, a diastolic blood pressure, a systolic blood pressure, a beat-to-beat blood pressure and a tonoarteriogram.

9. The wearable apparatus for measuring blood pressure signal according to claim 1, wherein the earphone body is at least one of an in-ear earphone body and an ear-hook earphone body.
